(19) United States
(12) Patent Application Publication
(10) Pub. No.: US 2008/0091111 A1
(43) Pub. Date: Apr. 17, 2008

(54) Sphygmomanometer Of Multi Functional

(76) Inventor: Jae Woo Shin, Seoul (KR)

Correspondence Address: OSHA LIANG L.L.P., 1221 MCKINNEY STREET, SUITE 2800, HOUSTON, TX, 77010, US

(21) Appl. No.: 11/658,194

(22) PCT Filed: Jul. 7, 2005
(86) PCT No.: PCT/KR05/02177

§ 371 Date: Jan. 22, 2007

(30) Foreign Application Priority Data
Jul. 23, 2004 (KR) 10-2004-0057649

(51) Int. Cl. A61B 5/0205

(52) U.S. Cl. 600/484

(57) ABSTRACT

Disclosed is a multifunctional sphygmomanometer. The multifunctional sphygmomanometer comprises a blood pressure measurement pressing part provided around the arm of a human body and receiving air, thereby measuring blood pressure; a respiration measurement pressing part provided around the abdomen of the human body and receiving air, thereby measuring respiration and enabling blood pressure reduction training; and a main body capable of supplying and exhausting air to the blood pressure measurement or respiration measurement pressing part, displaying the measured blood pressure or respiration or pulse, and controlling the respiration measurement pressing part to be changed for the blood pressure reduction training. Accordingly, it is possible to measure blood pressure and implement blood pressure reduction training in a simple and easy way at the same time since it is possible to measure blood pressure and respiration by using a pressing part connector and a pressure measurement method used in a conventional arm-type sphygmomanometer as they are, and it is possible to measure blood pressure and implement blood pressure reduction training with a minimum additional cost since a separate amplifying circuit or sensor for measuring respiration is not needed at all by using a motor pump and a valve provided to a conventional sphygmomanometer.

### TECHNICAL FIELD

The present invention relates to a multifunctional sphygmomanometer, and more particularly, to a multifunctional sphygmomanometer which can measure blood pressure by using a pressing part and a blood pressure measurement method used in a conventional arm-type sphygmomanometer, convert its function to measure respiration by using a respiration measurement pressing part, and enables training for reducing blood pressure through a biofeedback function regarding the respiration.

### BACKGROUND ART

Hypertension is emerging as a serious problem enough to be regarded as one of three diseases having the highest fatality in the modern society.

Among hypertension, essential hypertension whose abnormality of temperament is not found is a stress related disorder, and its cause is known to lie mainly in a psychological trait and physiological predisposition. Traditional drug therapy accessing a physiological cause has a problem that it may give rise to side effects. Particularly, in case of light hypertension, since use of drugs is not much recommended due to their side effects, the light hypertension may be a target for a psychological treatment.

Recently, it is expected that a biofeedback training, which is so-called third generation therapeutics of doing a psychological treatment without using a drug, will be efficient.

Namely, a biofeedback training method known to reduce blood pressure includes galvanic skin reflex, electromyography, temperature feedback, and respiration.

Among these, particularly, in case of the respiration, a slow deep respiration method using the abdomen is known as the most efficient blood pressure reduction training.

Unlike breast respiration whose respiration amount is small, thereby reducing its efficiency, abdominal respiration using the abdomen helps relaxation because oxygen and carbon dioxide can be exchanged most efficiently with the minimum efforts.

It is using a principle in which when deep respiration using the abdomen continues while suppressing respiration using the chest to the maximum, a respiration speed falls, as a result, a hear beat declines, and ultimately blood pressure is reduced.

Accordingly, since the respiration biofeedback training constructed as above can bring a meaningful effect to reduce blood pressure, it may be applied as a complementary or ancillary treatment to traditional drug therapy.

Conventional respiration training apparatus is constructed to measure respiration by using a strain gauge which is an elastic belt sensor.

In case of the strain gauge, it has disadvantages that its price is comparatively high, its life span is short because an external force is directly applied to a sensor, and its circuit configuration gets complicated.

Accordingly, although a corresponding function is included in a sphygmomanometer, there is another disadvantage that a separate sensor connecting terminal, circuit configuration and a separate method for zero-point adjustment are needed.

Since completely independent separate apparatus is needed to integrate apparatus having a blood pressure reduction training function through respiration measurement into a sphygmomanometer due to the reasons as described above, very high additional cost occurs.

### DISCLOSURE OF INVENTION

Technical Problem

The present invention is conceived to solve the above problems. Thus, the present invention provides a multifunctional sphygmomanometer which can measure blood pressure and implement blood pressure reduction training in a simple and easy way since it is possible to measure blood pressure and respiration by using a pressing part connector and a pressure measurement method used in a conventional arm-type sphygmomanometer as they are.

The present invention also provides a multifunctional sphygmomanometer which can measure blood pressure and implement blood pressure reduction training with a minimum additional cost since a separate amplifying circuit or sensor for measuring respiration is not needed at all by using a motor pump and a valve provided to a conventional sphygmomanometer.

Technical Solution

An object of the present invention is to provide a multifunctional sphygmomanometer which can measure blood pressure and implement blood pressure reduction training in a simple and easy way since it is possible to measure blood pressure and respiration by using a pressing part connector and a pressure measurement method used in a conventional arm-type sphygmomanometer as they are.

Also, another object of the present invention is to provide a multifunctional sphygmomanometer which can measure blood pressure and implement blood pressure reduction training with a minimum additional cost since a separate amplifying circuit or sensor for measuring respiration is not needed at all by using a motor pump and a valve provided to a conventional sphygmomanometer.

Advantageous Effects

As described above, according to a multifunctional sphygmomanometer of the present invention, there is an effect that it is possible to measure blood pressure and implement blood pressure reduction training in a simple and easy way since it is possible to measure blood pressure and respiration by using a pressing part connector and a pressure measurement method used in a conventional arm-type sphygmomanometer as they are.

Also, according to the present invention, there is an effect that it is possible to measure blood pressure and implement blood pressure reduction training with a minimum additional cost since a separate amplifying circuit or sensor for measuring respiration is not needed at all by using a motor pump and a valve provided to a conventional sphygmomanometer.

### BRIEF DESCRIPTION OF THE DRAWINGS

FIG. 1 is a state diagram illustrating providing of a respiration measurement pressing part and its connection to a main body according to the present invention;

FIG. 2 is a block diagram of a multifunctional sphygmomanometer system according to the present invention;

FIG. 3 is a configuration diagram of a respiration measurement pressing part according to the present invention;

FIG. 4 is a flow chart illustrating a blood pressure reduction training order according to the present invention;

FIG. 5 is a step flow chart of blood pressure reduction training according to the present invention;

FIG. 6 is a pressure change diagram inside a respiration measurement pressing part by a training order of a blood pressure reduction training mode according to the present invention; and

FIG. 7 is a screen configuration diagram of an LCD in an output part in a blood pressure measurement mode and blood pressure reduction training mode.

10; multifunctional sphygmomanometer 20; main body 21; pressure sensor

22; amplifier 30; control part 31; motor pump

32; exhaust valve 33; input part 34; button

35; output part 36; LCD 40; respiration measurement pressing part

50; pressing part 51; tube 52; port

53; connection part 60; belt 61; connection buckle

62; length adjustment ring 63; belt connection plate

70; blood pressure measurement pressing part

80; LCD in blood pressure measurement mode

81; dilator blood pressure display part

82; diastole blood pressure display part

83; pulse display part 84; pressure display part

85; exhaust display part

90; LCD in blood pressure reduction training mode

91; training time display part 92; respiration size display part

93; inspiration guide 94; expiration guide

95; inspiration signal display part 96; expiration signal display part

B; abdomen

MODE FOR THE INVENTION

A preferred first technical solution means of the present invention provides a multifunctional sphygmomanometer comprising: a blood pressure measurement pressing part provided around the arm of a human body, and receiving air, thereby measuring blood pressure; a respiration measurement pressing part provided around the abdomen of the human body, receiving air, thereby measuring respiration and enabling blood pressure reduction training; and a main body capable of supplying and exhausting air to the blood pressure measurement or respiration measurement pressing part, displaying the measured blood pressure or respiration or pulse, and controlling the respiration measurement pressing part to be changed for the blood pressure reduction training.

It is efficient that an example of a first configuration for efficiently embodying the purpose of the present invention includes the main body comprising, a power part supplying power to the main body; an input part capable of setting and selecting an operational function of the main body; an output part displaying blood pressure and respiration measured by the blood pressure measurement or respiration measurement pressing part, and blood pressure reduction training time and an operational function mode inputted by the input part; a tube connector connecting a tube of the blood pressure measurement or respiration measurement pressing part; an internal motor pump supplying air to the blood pressure measurement or respiration measurement pressing part; a pressure sensor measuring a change of the air pressure inside the blood pressure measurement or respiration measurement pressing part and converting the same into an electric signal; an exhaust valve capable of exhausting air inside the blood pressure measurement or respiration measurement pressing part; and a control part controlling opening/closing of the exhaust valve and an operation of the motor pump according to the function selected by the input part, and receiving a measurement value of the change of the air pressure change from the pressure sensor and transmitting the received measurement value to the output part to output the same.

It is efficient that an example of a second configuration for efficiently embodying the purpose of the present invention includes the output part which is provided with any one between a display window for visually displaying the operational function of the main body inputted by the input part and the measurement value of the air pressure change transmitted by the control part and a speaker for auditory indication thereof.

It is efficient that an example of a third configuration for efficiently embodying the purpose of the present invention includes the blood pressure measurement or respiration measurement pressing part comprising, a pressing part filled with air injected via the tube connector; a tube connecting the tube connector and the pressing part, thereby supplying or exhausting the air; and a belt enabling the blood pressure measurement or respiration measurement pressing part to be provided around the arm or abdomen of the human body.

It is efficient that an example of a forth configuration for efficiently embodying the purpose of the present invention further includes an amplifier amplifying a signal of the pressure sensor to transmit the same to the control part provided between the pressure sensor and the control part.

It is efficient that an example of a fifth configuration for efficiently embodying the purpose of the present invention includes the motor pump and the exhaust valve provided to be capable of supplying or exhausting air by a manual operation selectively.

A conventional arm-type sphygmomanometer comprises blood pressure measurement pressing part for being provided around the forearm, a main body, and a tube connecting the blood pressure measurement pressing part and the main body, in which the main body is provided with a tube connector for connecting the blood pressure measurement pressing part.

Air is injected into the blood pressure measurement pressing part provided around the forearm through the tube connector, so as to sense an air pressure change, thereby computing blood pressure.

When measuring blood pressure, a multifunctional sphygmomanometer according to the present invention connects a blood pressure measurement pressing part to a main body, thereby measuring the blood pressure, and when doing blood pressure reduction training, the multifunctional sphygmomanometer according to the present invention removes the blood pressure measurement pressing part from the main body and instead, provides a respiration measurement pressing part to the main body, thereby enabling abdominal respiration to be measured.

This uses that a volume change occurs at the chest and the abdomen of a human body when breathing (inspiration and expiration). If supplying a certain amount of air to the respiration measurement pressing part serving as an inflatable bag shielded not to leak inside air and providing the respiration measurement pressing part around a user's abdomen part, an abdominal volume changing movement that occurs while the user breathes changes the pressure inside the respiration measurement pressing part, which is transmitted to a pressure sensor inside the main body, thereby enabling respiration to be measured via a changed amount of the pressure. Also, it is possible to do blood pressure reduction training by using this.

Hereinafter, the present invention will be described in detail with reference to the accompanying drawings.

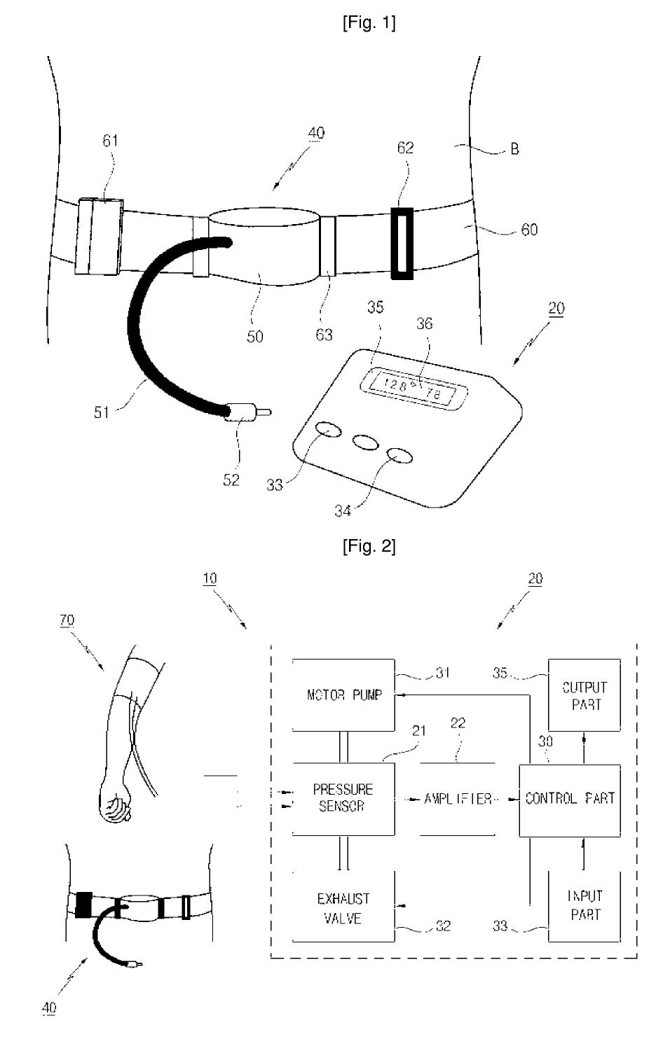

FIG. 1 is a state diagram illustrating providing of a respiration measurement pressing part and its connection to a main body according to the present invention. Referring to FIG. 1, a belt 60 enabling a respiration measurement pressing part 40 to be provided around the abdomen of a human body is provided by a connection buckle 61 and adjusted to be fit around the abdomen by using a length adjustment ring 62.

It is efficient that the belt 60 is formed of inelastic material.

A tube 51 provided at a pressing part 50 is connected to a main body 20 after providing the respiration measurement pressing part 40 around the abdomen.

It is preferable that the tube is formed of silicon material, and a respiration measurement pressing part 70 of FIG. 2 is also formed of silicon material.

A port 52 is provided to one end portion of the tube 51, and the preparations to measure respiration is over by inserting the port 52 into a tube connector (not illustrated) of the main body 20. At this time, attention should be paid not to bend the tube 51 since air injection and respiration measurement is impossible if the tube 51 is bent.

An input part 33, that is, a plurality of setting and input buttons 34 are provided on one side of the main body 20, and an output part 35, that is, an LCD 36 displaying contents regarding an operational function of the main body 20 that a user has inputted in the input part 34, information on respiration measured by the respiration measurement pressing part 40, and blood pressure reduction training is provided on the other side.

At this time, since a mode conversion button 34 enabling a conversion into a blood pressure measurement mode or a respiration measurement mode is included in the input part 33, it becomes possible to change a function of a multifunctional sphygmomanometer according to the present invention on the basis of a user's selection.

Also, while the output part 35 may be composed of a display window such as the LCD 36 visually displaying, it is preferable to use a speaker (not illustrated) for auditory indication at the same.

Also, although not illustrated, a power part (not illustrated) supplying power is provided inside the main body 20 for driving thereof.

Power for driving the main body 20 may be supplied via a power line (not illustrated) connected to an external socket, or may be supplied by a portable battery (not illustrated).

FIG. 2 is a block diagram of a multifunctional sphygmomanometer system according to the present invention. Referring to FIG. 2, in case of measuring blood pressure, a port of a tube (not illustrated) provided at the blood pressure measurement pressing part 70 is inserted into a tube connector (not illustrated) of the main body 20 after providing the blood pressure measurement pressing part 70 around the forearm of a user. In case of doing a respiration measurement or blood pressure reduction training, the respiration measurement pressing part 40 is provided around the abdomen of the user after removing the blood pressure pressing part 70 and also, the port 52 of the tube 51 is inserted into the tube connector (not illustrated) of the main body 20.

A pressure sensor 21 measuring an amount of air pressure inside the blood pressure measurement or respiration measurement pressing part 70 or 40 and converting a measured value into an electric voltage signal, an amplifier 22 amplifying the voltage signal converted by the pressure sensor 21, and a control part 30 controlling the voltage signal amplified by the amplifier 22 to be outputted via the output part 35 are provided inside the main body 20.

Also, the control part 30 controls an operation of a motor pump 31 and opening/closing of an exhaust valve 32 in accordance with a function set and selected by the user via the input part 33.

Since the tube connector (not illustrated) connecting the tube 51 of the blood pressure measurement or respiration measurement pressing part 70 or 40 and the pressure sensor 21, the motor pump 31 and the exhaust valve 32 provided inside the main body 20 are connected to one another, injection of air into the blood pressure measurement or respiration measurement pressing part 70 or 40 and maintenance of the air pressure therein, and pressure measurement are all implemented via the tube connector (not illustrated).

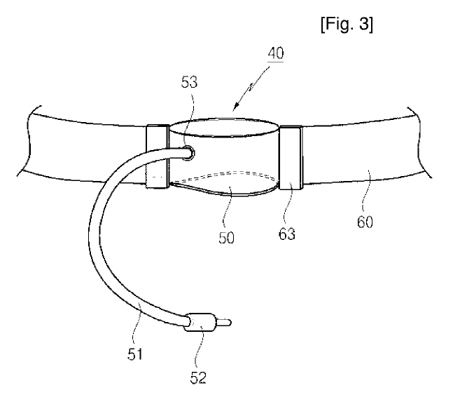

FIG. 3 is a configuration diagram of a respiration measurement pressing part according to the present invention. Referring to FIG. 3, all parts except a connection part 53 are shielded not to leak air when the air is injected into the respiration measurement pressing part 40.

The pressing part 50 formed of flexible plastic vinyl material is connected to the belt 60 via a pair of belt connection plates 63, the tube 51 starting from the connection part 53 is configured to be approximately 50 to 60 cm long and the port 52 is provided at the end portion of the tube 51, thereby being provided to the tube connector (not illustrated) of the main body 20.

Hereinafter, an operation of the present invention constructed such above will be described. While a drawing about use of the blood pressure measurement pressing part is omitted in the illustrated drawings, it does not affect the characteristics of the present invention so much. Also, since it is a conventional method, the drawing related thereto is omitted herein.

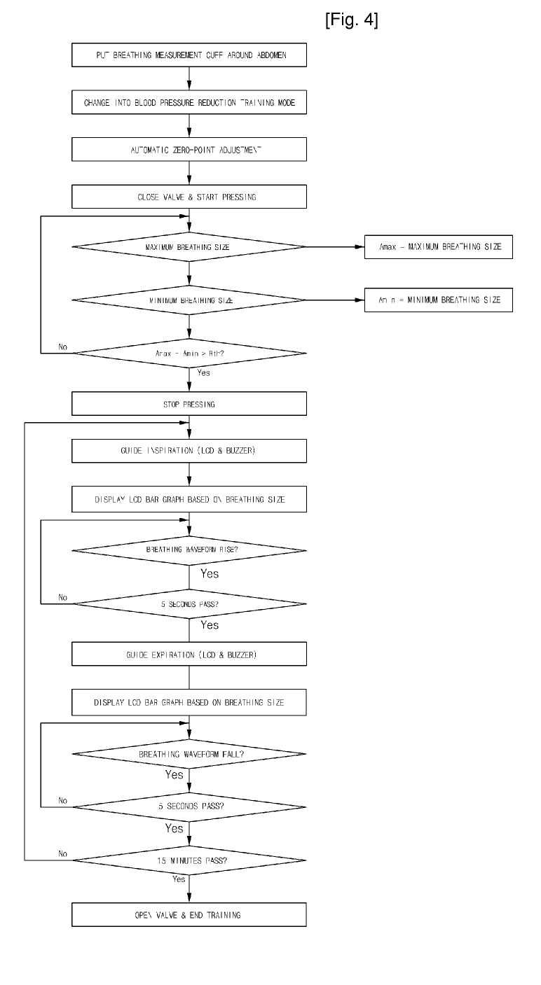

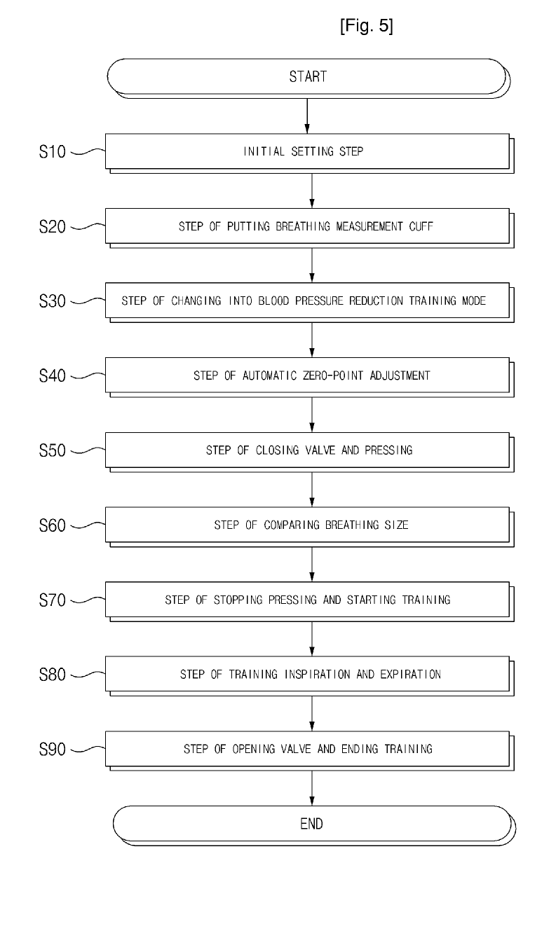

FIG. 4 is a flow chart illustrating a blood pressure reduction training order according to the present invention, and FIG. 5 is a step flow chart of blood pressure reduction training according to the present invention.

Amax indicates a maximum respiration size, Amin a minimum respiration size, and Rth a standard respiration size.

Referring to FIGS. 4 and 5, in order to perform blood pressure reduction training, firstly, the blood pressure measurement pressing part 70 is removed to provide the respiration measurement pressing part 40 around the abdomen, and the tube 51 is connected to the tube connector (not illustrated) of the main body 20 (S20).

A user can input various settings (an initial setting step such as setting of a training time, S10) regarding blood pressure reduction training before wearing the respiration measurement pressing part 40. Or, the user may use a blood pressure training mode which is basically set when the product is released from a factory.

In case of using the blood pressure reduction training mode basically set in release of the product, the initial setting step S10 is omitted. Hereinafter, use of the basic-set mode in release of the product will be described.

If pressing a mode conversion button among a plurality of buttons 34 of the input part 33 provided on the outside surface of the main body 20, a blood pressure measurement mode is changed into a blood pressure reduction training mode (S30).

At this time, when using the blood pressure measurement mode or when using the blood pressure reduction training mode, an automatic zero-point adjustment is implemented before injecting air into the blood pressure measurement or respiration measurement pressing part 70 or 40 whereby a process of adjusting a reference value to 0 continues for 1 or 2 seconds (S40).

The exhaust valve 32 is closed to inject air into the respiration measurement pressing part 70 after the automatic zero-point adjustment (S40) is over, and the pressure is slowly added by using the motor pump 31 (S50).

A user continues to breathe while pressing, and the pressure sensor 21 detects a maximum respiration size (Amax) in a state when the user breathes out and a minimum respiration size (Amin) in a state when the user breathes in via a pressure change of air inside the respiration measurement pressing part 70 according to the size of respiration.

If a difference value between the maximum value and the minimum value exceeds a standard respiration size (Rth) when the difference value is compared with the standard respiration size (Rth) (S60), the control part 30 stops an operation of the motor pump 31 to stop pressing, and gets down to training preparations (S70).

The goal of respiration training for reducing blood pressure, set in the present invention, is to breathe in five seconds and breathe out five seconds for 15 minutes (S80). Also, the user may separately set it in the initial setting step (S10).

Inspiration is guided in initial training. At this time, the user receives an indication to inspire by simultaneously generating a human voice or a buzzer sound and a display via the output part 35, that is, the LCD 36 and the speaker (not illustrated).

When the inspiration is guided, the user has to suppress his/her chest movement to the maximum and breathe in by using the abdomen only.

At this time, it is better to display an inspiration sate of respiration on the LCD 36 by using a bar graph (92 of FIG. 7), so as to enable the user to feedback his/her abdominal movement to induce a maximum abdominal movement.

Since inspiration is a process of enabling air to flow into the lung, the abdomen swells up, thereby increasing pressure inside the respiration measurement pressing part 40.

If the pressure continuously increases for five seconds, inspiration training is successfully achieved. Therefore, after this, an expiration guide is displayed via the LCD 36 and a buzzer sound or a human voice.

Since expiration is that air gets out of the lung, the abdomen is constricted, thereby decreasing pressure inside the respiration measurement pressing part 40.

If the pressure continuously decreases for five seconds, expiration training is successfully achieved. Therefore, after this, inspiration is guided again.

If training as above continues for 15 minutes, the training automatically stops and the exhaust valve 32 rapidly exhausts all air inside the respiration measurement pressing part 40 whereby the training ends (S90).

At this time, unless pressure inside the respiration measurement pressing part 40 increases (decreases), that is, unless a respiration waveform rises (falls), the control part 30 waits until the respiration waveform rises (falls).

Also, at this time, unless a respiration waveform rises (falls) even aft a certain time elapses by setting a standby time, it may be possible to give an inspiration (expiration) guide again.

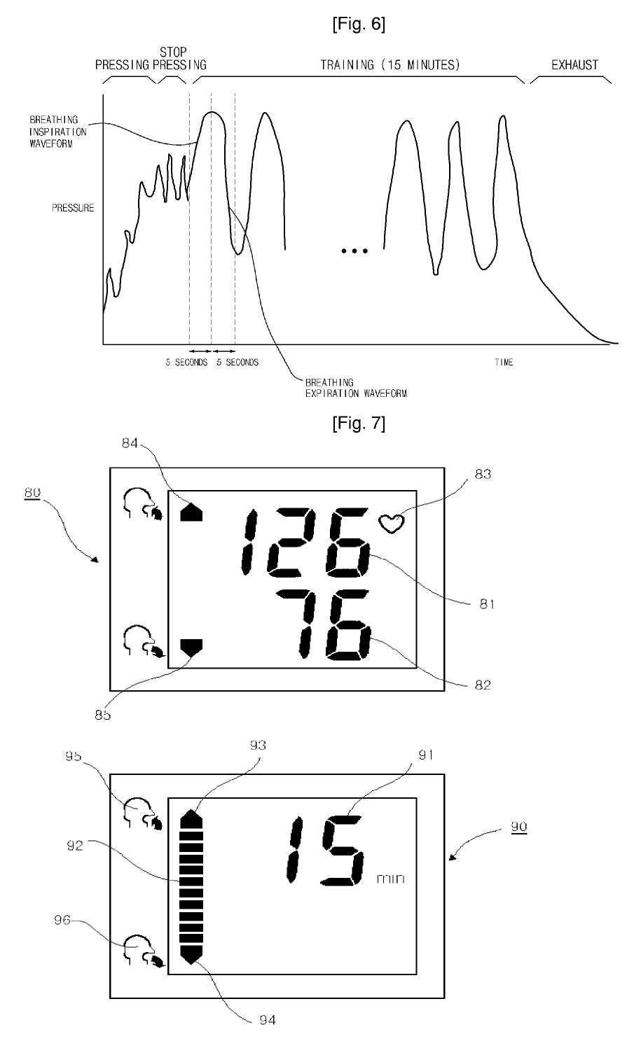

FIG. 6 is a pressure change diagram inside a respiration measurement pressing part according to a training order of a blood pressure reduction training mode. Referring to FIG. 6, firstly, a blood pressure reduction training mode is selected and air is injected into the respiration measurement pressing part 40 by using the motor pump 31 after an automatic zero-point adjustment is over. This causes pressure to continue to rise.

If an amount of air enough to measure respiration of a user by using the respiration measurement pressing part 40 is injected, the control part 30 stops an operation of the motor pump 31 to stop pressing and blood pressure reduction training gets started.

If five-second-interval inspiration and expiration is successfully achieved via continuous abdominal movements, the control part 30 opens the exhaust valve 32 to exhaust air rapidly, thereby ending training and the pressure inside the respiration measurement pressing part 40 falls down to 0 mmHg.

FIG. 7 is a screen configuration diagram of an LCD in an output part in a blood pressure measurement mode and a blood pressure reduction training mode. Referring to FIG. 7, since the LCD 80 is formed of largely two portions in a blood pressure measurement mode, a dilator and a diastole blood pressure 81 and 82 are displayed after measurement of blood pressure.

Since an automatic zero-point adjustment process is essential before measurement, an exhaust display part 85 is provided to display an exhaust state of the exhaust valve 32.

If blood pressure measurement starts, the control part 30 operates the motor pump 31, thereby supplying air to the blood pressure measurement pressing part 70. At this time, a pressure display part 84 is provided to display a pressure state and a pulse generation state is displayed via a pulse display part 83 while measuring pulses.

Unlike a blood pressure measurement mode, in case of implementing a blood pressure reduction training mode, numbers and signals displayed on the screen of an LCD 90 serve respectively different roles.

First, a portion used to display the dilator blood pressure 81 is used as a training time display part 91, so as to let a user know how long a training time has elapsed.

Also, display parts used as the pressure display part 84 and the exhaust display part 85 in a blood pressure measurement mode are used as inspiration guide and expiration guide 93 and 94, respectively.

Also, respiration size display parts 92 formed of a plurality of rectangles are provided between an inspiration signal display part 95 and an expiration signal display part 94, and thereby, display a user's abdominal movement state to provide a visual biofeedback.

At this time, a plurality of rectangular signals is used to display the user's abdominal movement sate, but it is preferable to enable the user to have a kind of fun in blood pressure reduction training by displaying the state in an entertaining character's movements or the like.

In the present invention, it is described that an interval between inspiration and expiration in blood pressure reduction training is five seconds and the total training time is 15 minutes, but it is preferable to enable each training time to be added and subtracted in accordance with the user's setting.

In the present invention, it is described that power is supplied to drive a main body and thereby, a control part automatically controls an operation of a motor pump and opening/closing of an exhaust valve. However, if there is no power supplied to the main body, it is preferable that the motor pump and the exhaust valve comprise a manual air pump and a manual exhaust valve which can supply and exhaust air manually.

It will be apparent to those skilled in the art that various modifications and variations can be made in the present invention without departing from the spirit or scope of the inventions. Thus, it is intended that the present invention covers the modifications and variations of this invention provided they come within the scope of the appended claims and their equivalents.

### INDUSTRIAL APPLICABILITY

A multifunctional sphygmomanometer according to the present invention can measure blood pressure by using a pressing part and a pressure measurement method used in a conventional arm-type sphygmomanometer and convert its function, thereby capable of measuring respiration via a respiration measurement pressing part and implementing training for reducing blood pressure via a biofeedback function to the respiration.

1. A multifunctional sphygmomanometer comprising:
a blood pressure measurement pressing part provided around the arm of a human body and receiving air, thereby measuring blood pressure;
a respiration measurement pressing part provided around the abdomen of the human body and receiving air, thereby measuring respiration and enabling blood pressure reduction training; and
a main body capable of supplying and exhausting air to the blood pressure measurement or respiration measurement pressing part, displaying the measured blood pressure or respiration or pulse, and controlling the respiration measurement pressing part to be changed for the blood pressure reduction training.

2. The multifunctional sphygmomanometer according to claim 1, wherein the main body includes:
a power part supplying power to the main body; an input part capable of setting and selecting an operational function of the main body;
an output part displaying blood pressure and respiration measured by the blood pressure measurement or respiration measurement pressing part, and a blood pressure reduction training time and an operational function mode inputted by the input part;
a tube connector connecting a tube of the blood pressure measurement or respiration measurement pressing part; an internal motor pump supplying air to the blood pressure measurement or respiration measurement pressing part;
a pressure sensor measuring a change of air pressure inside the blood pressure measurement or respiration measurement pressing part and converting the same into an electric signal;
an exhaust valve capable of exhausting air inside the blood pressure measurement or respiration measurement pressing part; and a control part controlling opening/closing of the exhaust valve and an operation of the motor pump according to a function selected by the input part, receiving a measurement value of the air pressure change from the pressure sensor and transmitting the received measurement value to the output part to output the same.

3. The multifunctional sphygmomanometer according to claim 2, wherein the output part is provided with any one between a display window for visually displaying the operational function of the main body inputted by the input part and the measurement value of the air pressure change transmitted by the control part and a speaker for auditory indication thereof.

4. The multifunctional sphygmomanometer according to claim 1, wherein the blood pressure measurement or respiration measurement pressing part includes:
a pressing part filled with air injected via the tube connector;
a tube connecting the tube connector and the pressing part, thereby supplying or exhausting air; and
a belt enabling the blood pressure measurement or respiration measurement pressing part to be provided around the arm or abdomen of the human body.

5. The multifunctional sphygmomanometer according to claim 2, wherein an amplifier amplifying a signal of the pressure sensor to transmit the same to the control part is further provided between the pressure sensor and the control part.

6. The multifunctional sphygmomanometer according to claim 2, wherein the motor pump and the exhaust valve are provided to be capable of supplying or exhausting air by a manual operation selectively.

7. The multifunctional sphygmomanometer according to claim 2, wherein the blood pressure measurement or respiration measurement pressing part includes:
a pressing part filled with air injected via the tube connector;
a tube connecting the tube connector and the pressing part, thereby supplying or exhausting air; and
a belt enabling the blood pressure measurement or respiration measurement pressing part to be provided around the arm or abdomen of the human body.
